(19) United States
(12) Patent Application Publication
(10) Pub. No.: US 2006/0247604 A1
(43) Pub. Date: Nov. 2, 2006

(54) Lavage chair and method of use

(76) Inventor: Roy R. Bruno, Austin, TX (US)

Correspondence Address: ERIC B. MEYERTONS;MEYERTONS, HOOD, KIVLIN,, KOWERT & GOETZEL, P.C., P.O. BOX 398, AUSTIN, TX, 78767-0398, US

(21) Appl. No.: 11/118,123

(22) Filed: Apr. 29, 2005

(51) Int. Cl. A61M 1/00

(52) U.S. Cl. 604/540

(57) ABSTRACT

A lavage chair may support a human in an at least partially supine position. The lavage chair may be portable. The lavage chair may include a back connected to the base. The base may include a front portion. The lavage chair may be removably coupled to a toilet or to a waste container. In some embodiments, an extendable member may be coupled to the base. The extendable member may support a fluid source (e.g., an enema kit). A method of use may include allowing fluid to flow from the fluid source, through the front portion of the base, and into the colon or the vagina of the human. Effluent from the colon or the vagina of the human may flow through a drain in the base to the toilet or to the waste container.

### BACKGROUND

1. Field of Invention

The present invention generally relates to a lavage chair that supports a human in an at least partially supine position. An embodiment of the invention includes a portable lavage chair that removably couples to a toilet.

2. Description of Related Art

Medical procedures, routine care of mobility impaired persons, or personal hygiene may involve lavage (procedures that include rinsing, and/or removal of waste material, from the colon and/or vagina of a human. Devices may facilitate injection of liquid into the colon by way of the anus and/or egress from the colon of fecal material along with the injected liquid. U.S. Pat. No. 4,628,554 to Copp, which is incorporated herein by reference, describes a support for the prone back during an upper enema, in which the legs are folded, with the feet resting on footrests and the buttocks adjacent to an effluent shield which stops the effluent and allows it to drop into a toilet, on which the lower part of the support rests. U.S. Pat. No. 4,682,979 to Girouard, which is incorporated herein by reference, describes colon washing apparatus and methods wherein water flow to the colon may be by gravity flow or by pumped flow.

### SUMMARY

A lavage chair for a human may include a base and a back connected to the base. The lavage chair may be portable. The back and the base of the lavage chair may support a human in an at least partially supine position. In some embodiments, the back is connected to the base at an angle of at least 10° between the back and a surface upon which the lavage chair rests. In certain embodiments, the lavage chair includes an extendable member configured to support a fluid source at a height sufficient to allow fluid from the fluid source to enter the colon or the vagina of the at least partially supine human during use.

The lavage chair may include a front portion coupled to the base. The front portion may include an opening to allow fluid to flow from the fluid source, through the opening, and to the colon or the vagina of the at least partially supine human during use. The lavage chair may be removably coupled to a toilet or to a waste container. In some embodiments, the lavage chair includes a drain to allow effluent to move from the colon or the vagina of the at least partially supine human to the toilet or to the waste container during use.

Certain embodiments of lavage chairs may include combinations of the above features, and/or combinations of features set forth in this patent. Certain lavage methods may include use of such features.

### BRIEF DESCRIPTION OF THE DRAWINGS

Advantages of the present invention will become apparent to those skilled in the art with the benefit of the following detailed description and upon reference to the accompanying drawings in which:

FIG. 1 depicts a perspective view of an embodiment of a lavage chair.

FIG. 2 depicts a perspective view of a portion of the extendable member of embodiment of the lavage chair shown in FIG. 1.

FIG. 3 depicts a front view of an embodiment of a lavage chair.

FIG. 4 depicts a cutaway view of the front portion of an embodiment of a lavage chair.

FIG. 5 depicts a side view of an embodiment of a lavage chair, with a user positioned on the lavage chair and the lavage chair removably coupled to a toilet.

FIG. 6 depicts a front view of an embodiment of a lavage chair removably coupled to a waste container.

FIG. 7 depicts a perspective view of an embodiment of a waste container used with a lavage chair.

FIG. 8 depicts a perspective view of an embodiment of a lavage chair with a back folded toward the front portion and an extendable member retracted into the front portion.

FIG. 9 depicts a perspective view of an embodiment of a lavage chair removably coupled to a waste container.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION

Health care and/or personal hygiene in a residential or institutional setting may include lavage of one or more internal organs. As used herein, “lavage” generally refers to the washing out of an organ of a human. For example, “lavage” may refer to therapeutic or hygienic cleansing of the colon or the vagina, as achieved with an intestinal enema or a vaginal douche. As used herein, “chair” generally refers to any of various devices with a seat portion that hold up or support a human. As disclosed herein, a lavage chair may include a fluid source to provide safe, effective, comfortable, and/or discreet internal cleansing. In some embodiments, a fluid source may be used to provide external cleansing as well.

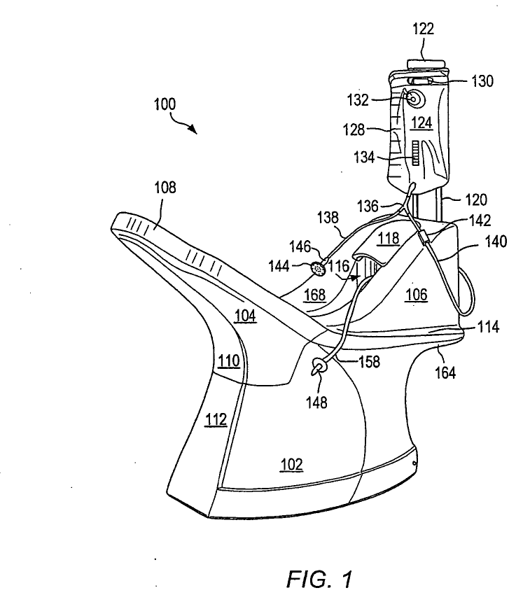

FIG. 1 depicts an embodiment of lavage chair 100. In some embodiments, lavage chair 100 is a single piece of molded material (e.g., molded plastic, rubber, and/or polyethylene). In certain embodiments, lavage chair 100 includes two or more coupled or connected pieces. As used herein, “coupled” pieces may be used operatively with each other, or joined or linked together, with or without one or more intervening members. Pieces that are “removably coupled” may be coupled and then uncoupled without permanently altering the coupled pieces. As used herein, “connected” pieces may be joined or linked together. In some embodiments, pieces that are described as “connected” or “coupled” may be a single piece (e.g., molded as a single piece).

Lavage chair 100 is typically designed to support the head, neck, back, buttocks, and/or feet of an at least partially supine human during lavage. Lavage may include, but is not limited to, administration of an enema or a douche. As used herein, “enema” generally refers to the injection of liquid into the colon through the anus for cleansing, for stimulating evacuation of the bowels, or for other therapeutic or diagnostic purposes. “Douche” generally refers to a jet or current of fluid directed against a part or into a cavity of the body (e.g., vagina) for cleansing. As used herein, “support” of a human is generally used to mean to independently and ergonomically bear the weight of the human in a stable and/or comfortable manner. Thus, lavage chair 100 is a stand-alone unit that rests on a surface (e.g., a floor) without leaning on or being propped up by external mechanisms. As used herein, “at least partially supine human” generally refers to a human lying on the back or with the face upward (e.g., reclining; leaning or sloping backward) such that an angle between the back of the user and the surface on which lavage chair 100 rests is at most 90°.

As used herein, “user” generally refers to the human in an at least partially supine position who has undergone, is undergoing, or will undergo lavage seated in lavage chair 100. As used herein, “operator” generally refers to someone who transports and/or operates lavage chair 100. In some cases, the user and the operator are the same person. In other cases, the user and the operator are two different people. For example, the user may be a mobility impaired person and the operator may be a health care provider or someone who specializes in colon hydrotherapy.

In the following description, embodiments of lavage chair 100 will be described for use with an enema kit for cleansing of the colon. It is to be understood, however, that lavage chair 100 may also be used with a douche kit for cleansing of the vagina. As used herein, “enema kit” generally refers to a device intended to instill water or other fluids into the colon through a nozzle inserted into the rectum to promote evacuation of the contents of the lower colon. The device consists of a container for fluid coupled to the nozzle either directly or via one or more conduits. As used herein, “douche kit” generally refers to a device that is a container (e.g., a bag or a bottle) coupled to one or more conduits and a nozzle. After filling the bag or the bottle with fluid, the nozzle is used to direct a stream of the fluid into the vaginal cavity for cleansing of the vaginal cavity.

The angle between the back of user and the surface on which the lavage chair rests may be chosen to facilitate flow of fluid into the colon of the user and/or egress of effluent. As used herein, “effluent” is generally used to include fluid (e.g., bodily fluid and/or fluid from a fluid source) and/or fecal material from the colon of the user. An angle of incline of the user's back may promote intra-abdominal pressure that facilitates cleansing of the user's colon. In some embodiments, an angle between the back of the user and the surface on which the lavage chair rests may range from about 10° to about 80°, from about 20° to about 60°, or from about 25° to about 40°. The angle between the back of the user and the surface on which lavage chair 100 rests may range continuously or in discrete increments. In certain embodiments, the angle between the back of the user and the surface on which lavage chair 100 rests may be fixed. For example, the angle between the back of the user and the surface on which lavage chair 100 rests may be fixed at about 30°.

As depicted in FIG. 1, lavage chair 100 may include base 102 and back 104. In some embodiments, base 102 may be substantially hollow, thereby lowering the cost and lessening the weight of the lavage chair. In certain embodiments, base 102 may include structural components including, but not limited to, polymer foam or metal. For example, base 102 may be filled with polystyrene or polyurethane foam. In some embodiments, base 102 may be a container that can be filled with a fluid (e.g., water) for ballast and drained. Base 102 may include front portion 106. Back 104 may be connected to base 102. In some embodiments, back 104 is a backrest. Back 104 may be movable with respect to base 102 and/or front portion 106 of lavage chair 100. In some embodiments, back 104 may be rotatably connected (e.g., hinged) to base 102. Back 104 may be sized, shaped, and/or contoured to comfortably support the back of a user. Back 104 may include headrest 108. In some embodiments, at least a portion of back 104 may be cushioned. For example, a cushion may be removably coupled to headrest 108.

Back 104 may be connected to base 102 such that a portion of the back mates with a portion of the base to provide structural support to lavage chair 100. In some embodiments, a bottom edge of back 104 mates with a top edge of base 102. For example, reinforced portion 110 of back 104 may substantially mate with reinforced portion 112 of base 102, thereby providing additional strength to lavage chair 100. Reinforced portions 110, 112 may be regions of solid molded material (e.g., plastic or polyethylene). In some embodiments, one or more of reinforced portions 10, 112 may include additional interior support (e.g., polymer foam or a metal insert) or exterior support (e.g., a metal plate). Reinforced portions 110, 112 may be designed such that lavage chair 100 supports a user weighing at most 250 pounds, at most 275 pounds, or at most 300 pounds. In certain embodiments, back 104 may be reversibly locked to base 102 (e.g., proximate to reinforced portions 110, 112) with any device generally known in the art to inhibit unintentional movement of back 104 with respect to base 102. Locking devices may include, but are not limited to, metal hook and eye type fasteners, sliding bolts, and flexible hook and loop type fasteners.

Base 102 may include one or more footrests 114. Footrests 114 may include, but are not limited to, rails, ledges, and stirrups. In some embodiments, as depicted in FIG. 1, footrests 114 are an integral part of base 102. In certain embodiments, footrests 114 include one or more members coupled to base 102. Members of footrests 114 may be made of materials including, but not limited to, metal, plastic, or a combination thereof.

In some embodiments, front portion 106 is an integral part of base 102. In certain embodiments, front portion 106 may be connected to base 102. As depicted in FIG. 1, front portion 106 may include opening 116. Opening 116 may be shaped to accept fluid. For example, opening 116 may serve as a basin or a trough. Splashguard 118 may cover a portion of opening 116 to inhibit splashing of the user with effluent during use. In some embodiments, splashguard 118 is removably coupled to front portion 106 to facilitate cleaning of the splashguard and the front portion following use of lavage chair 100.

In some embodiments, extendable member 120 may be coupled to front portion 106. Extendable member may include handle 122. A height of extendable member 120 in a fully extended position may be from about 20-80 inches, from about 30-70 inches, from about 40-60 inches, or from about 45-55 inches. In an extended position, extendable member 120 may be used to support fluid source 124 at a height such that the force of gravity allows fluid to flow from the fluid source into the colon or the vagina of the user. In some embodiments, a height of extendable member 120 in a fully extended position may be about 50-70 inches. The height of extendable member 120 may be continuously or incrementally adjustable. In certain embodiments, extendable member 120 may be fully retractable into base 102 (e.g., into front portion 106 of the base). For example, extendable member 120 may include “telescoping” concentrically sized lengths, such that pushing unlocked handle 122 toward (or pulling the handle away from) base 102 or front portion 106 causes the concentrically sized lengths (e.g., cylindrical sections) to slide or pass one within another, thereby reducing (or increasing) the height of the extendable member. Extendable member 120 may be locked in a fully extended position and/or in a fully retracted position with, for example, a spring-loaded ball/detent mechanism.

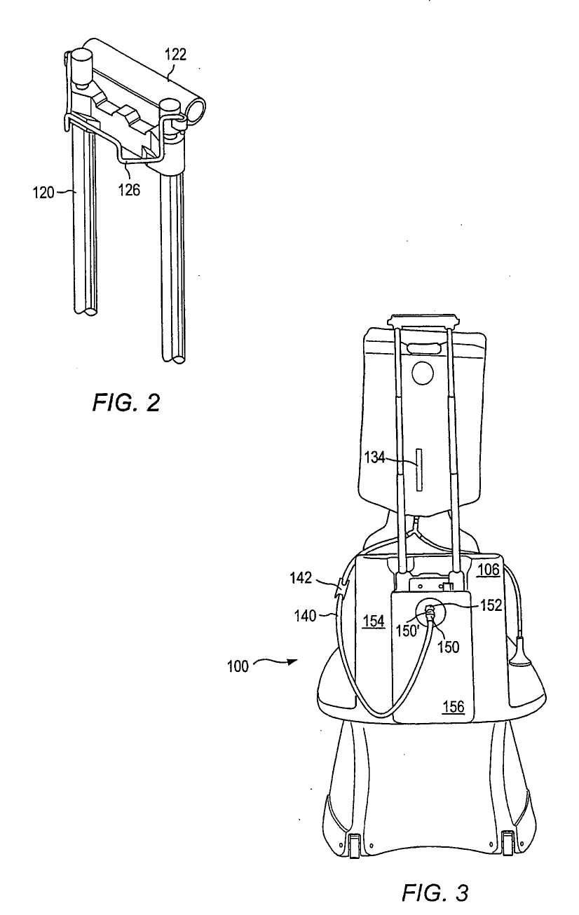

FIG. 2 depicts an embodiment of handle 122 of extendable member 120. In some embodiments, member 126 may be removably coupled to handle 122. Member 126 may be a wire hanger shaped and/or sized to support fluid source 124. In some embodiments, fluid source 124 may hang from member 126. For example, a handle of fluid source 124 may fit over member 126. Member 126 may allow fluid source 124 to be quickly coupled to and uncoupled from extendable member 120.

As depicted in FIG. 1, fluid source 124 may be a refillable container or a disposable kit (e.g., a disposable enema or douche kit). In some embodiments, fluid source 124 is a container that rests on a surface (e.g., a shelf) proximate to lavage chair 100. In certain embodiments, fluid source 124 is a flexible transparent or translucent bag. Fluid source 124 may be made of materials including, but not limited to, thermoplastic polyurethane. In some embodiments, fluid source 124 is a collapsible shower bag. In certain embodiments, fluid source 124 holds from about 1-5, or about 2-4, gallons of water. Fluid source 124 may include indicia 128 to indicate a volume of fluid in the fluid source. Dimensions of fluid source 124 may be, for example, about 10-15 inches by about 15-20 inches.

Fluid source 124 may be removably coupled to member 126. For example, member 126 may fit through opening 130 of fluid source 124 to support and elevate the fluid source during use. Dimensions of opening 130 may be, for example, about 1 inch by about 4 inches. Cap 132 may be coupled to a threaded opening in fluid source 124. Cap 132 may be uncoupled from fluid source 124 (e.g., removed from the threaded opening of the fluid source) to allow the fluid source to be filled with fluid. The opening may be, for example about 1-3 inches in diameter, or at least 2 inches in diameter. Cap 132 may be tethered to fluid source 124 to inhibit loss or misplacement of the cap.

Fluid source 124 may be filled with water or an aqueous solution (e.g., including medicinal or cleansing additives) of a desired temperature. The fluid may be prepared by the operator or purchased as a pre-mixed and/or pre-measured kit. Fluid source 124 may include temperature indicator 134 to allow assessment of a temperature of fluid in the fluid source. Temperature indicator 134 may be, for example, a liquid crystal temperature strip. Assessment of the temperature of fluid in fluid source 124 may inhibit burning or scalding of the user, since the fluid is often heated prior to use. In some embodiments, fluid source 124 may include a recirculating device or a mixing device designed to recirculate or to mix fluid in the fluid source. In certain embodiments, fluid source 124 may include a heating device designed to warm fluid in the fluid source to a desired temperature.

Fluid source 124 may be coupled to one or more conduits. As depicted in FIG. 1, fluid source 124 may be coupled to (e.g., threaded to) member 136. Member 136 may be a “T” or a “Y”. Member 136 may be coupled to conduits 138, 140. In an example, conduits 138, 140 are ½ inch outer diameter, ⅜ inch inner diameter vinyl (e.g., polyvinyl chloride) tubing, with a wall thickness of 1/16 inch or 3/32 inch. Conduits 138, 140 may range in length from about 12 inches to about 36 inches. For example, conduits 138, 140 may be about 24 inches in length. Conduit 138 and/or conduit 140 may be coupled to valve or regulator 142. Valve or regulator 142 may be, for example, a thumb clamp. Valve or regulator 142 may allow the user to regulate a flow rate or inhibit a flow of fluid from fluid source 124.

Conduit 138 may be used to provide fluid for exterior cleansing of a portion of the user's body. Conduit 138 may be coupled to dispensing member 144. Dispensing member 144 may be, for example, a nozzle, spigot, or sprayer. Fluid may flow from fluid source 124 through conduit 138 to dispensing member 144. Dispensing member 144 may include valve or regulator 146 to allow the user to regulate fluid flow through the member. In some embodiments, valve or regulator 146 may be a restrictor valve (e.g., pull-on, push-off type) or a push button valve. Dispensing member 144 may function as a bidet. That is, dispensing member 144 may allow fluid from fluid source 124 to be directed toward a portion of a user's body to allow external cleansing of the portion of the body. In some embodiments, fluid source 124 includes two compartments, such that fluid flowing through conduit 138 for external cleansing may be of a different composition than fluid flowing through conduit 140 for internal cleansing. In some embodiments, conduits 138, 140 may be coupled to separate fluid sources (e.g., two different fluid containers). In some embodiments, one or both of conduits 138, 140 are coupled to one or more backflow prevention valves to inhibit backflow of fluid to one or more fluid sources.

Conduit 140 may be used to provide fluid to the colon of the user. In some embodiments, conduit 140 may be coupled to dispensing member 148. Dispensing member 148 may be a nozzle designed to be inserted in the anus of the user. For example, dispensing member 148 may be selected from disposable or reusable rectal nozzles of various shapes and/or sizes. In some embodiments, fluid may flow from fluid source 124 through conduit 140 and dispensing member 148 into the colon of the user. In certain embodiments, as depicted in FIG. 3, conduit 140 may be removably coupled to connector 150. Connector 150 may be, for example, a quick connect.

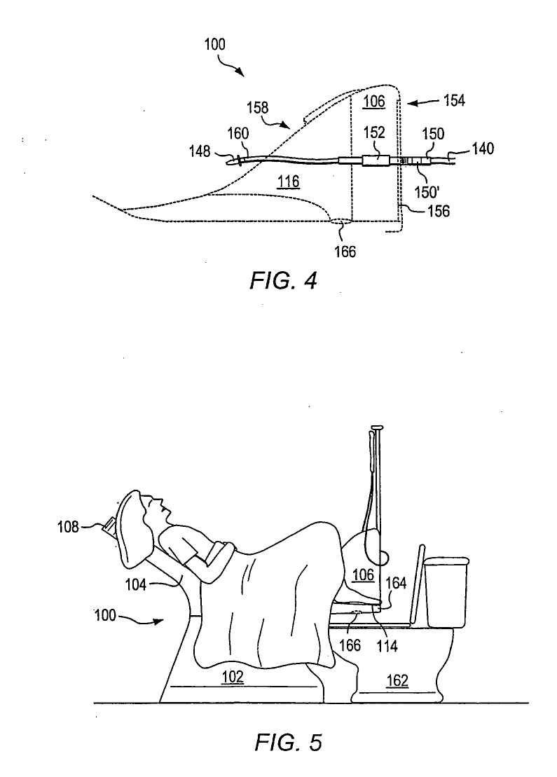

FIG. 4 depicts a cutaway view of front portion 106 of lavage chair 100. Connector 150 may be coupled to mating connector 150′. Connector 150′ may be coupled, with one or more intervening members, to a first end of valve or regulator 152 proximate front side 154 of lavage chair 100. In some embodiments, front side 154 may include removable cover 156, as depicted in FIG. 3. Connectors 150, 150′ may allow fluid to flow though the connectors while the connectors are coupled (e.g., mated) to each other. Thus, with connectors 150, 150′ coupled, fluid may flow from fluid source 124 through conduit 140 and connectors 150, 150′ through valve or regulator 152 toward opening 116 (e.g., toward back side 158 of front portion 106). Connectors 150, 150′ may inhibit fluid flow from conduits coupled to the connectors while the connectors are uncoupled. For example, with connectors 150, 150′ uncoupled, leakage of fluid from conduit 140 through connector 150 is inhibited.

In some embodiments, a first end of conduit 160 may be coupled to a second end of valve or regulator 152. Valve or regulator 152 may allow directional flow of fluid. In certain embodiments, valve or regulator 152 is a ball check valve. Valve or regulator 152 may allow flow of fluid in one direction. For example, valve or regulator 152 may allow fluid to flow from front side 154 toward back side 158 of front portion 106 (e.g., from the front side toward opening 116) while inhibiting flow of fluid from the back side toward the front side. That is, fluid may be allowed to flow away from fluid source 124 and inhibited from flowing toward the fluid source such that fluid flows from the fluid source through conduit 140, valve or regulator 152, and conduit 160. A second end of conduit 160 may be coupled to dispensing member 148. With dispensing member 148 inserted in the anus of the user, fluid may flow from fluid source 124 into the colon of the user. Valve or regulator 142 may be used to adjust a rate of fluid flow into the colon of the user.

FIG. 5 depicts lavage chair 100 removably coupled to toilet 162, with a user positioned on the lavage chair. Lavage chair 100 may be positioned in any orientation around toilet 162 such that ledge 164 extends over the bowl of the toilet and drain 166 (also depicted in FIG. 4) is positioned over the bowl. The user's back and head are resting on back 104 and headrest 108, respectively. The user's feet are resting on footrests 114 or on a superior surface of front portion 106, and the buttocks are positioned in seat portion 168 proximate opening 116 (an example of which is depicted in FIG. 1).

An angle of back 104 with respect to the surface on which lavage chair 100 rests may advantageously promote safe use of member 148. For example, a user lying flat may inadvertently insert a rectal nozzle too deeply and thereby irritate or damage the lining of the colon. However, an angle of back 104 of, for example, about 10°-80°, about 20°-60°, about 25°-40°, or about 30°, may allow more shallow penetration of member 148 into the user's rectal opening, thereby inhibiting damage or irritation of the wall of the colon.

Lavage chair 100 may be sized and/or shaped for use in a variety of spaces, such as a bathroom in a home, a bathroom in an institutional setting, or a bathroom stall. If desired, lavage chair 100 may be used in a bathtub or a shower. In an example, lavage chair 100 may have a length along the bottom of the base of less than about 40 inches (e.g., about 35 inches), a headrest height of less than about 50 inches (e.g., about 40 inches), and a total length of less than about 70 inches (e.g., about 55 inches). Ledge 164 (depicted in FIGS. 1 and 5) may have a height of about 20 inches (e.g., 18 inches) from the bottom of base 102, allowing use of lavage chair 100 with toilets of various designs. A sturdy lavage chair with smaller dimensions may be advantageously used in smaller spaces (e.g., smaller bathrooms) than a lavage chair with larger dimensions. Thus, a lavage chair with a smaller footprint (or area on a surface covered by the base of the lavage chair) is more versatile and may be used in more locations than a lavage chair with a larger footprint.

The operator may allow fluid to flow into the colon of the user through member 148 as desired. Effluent from the user's colon may be allowed to flow into opening 116 in front portion 106 of lavage chair 100. In some embodiments, the operator or the user may choose to allow effluent to flow from the user's colon with member 148 positioned in the anus of the user. In certain embodiments, the operator or the user may remove member 148 from the user's anus before effluent flows from the user's colon.

Effluent that flows from the user's colon into opening 116 may flow downward toward drain 166. In some embodiments, drain 166 may have a funnel-like shape. Drain 166 may have an opening of, for example, from about 2-5 inches in diameter, or about 3.5 inches in diameter. In certain embodiments, as depicted in FIG. 5, lavage chair 100 may be removably coupled to (e.g., positioned proximate to) toilet 162. With ledge 164 extending over the bowl of toilet 162, effluent may flow through drain 166 and into the bowl of the toilet. In some embodiments, lavage chair 100 may be connected to toilet 162 to inhibit unintentional movement of the lavage chair during use. For example, lavage chair 100 may be connected to toilet 162 with a strap.

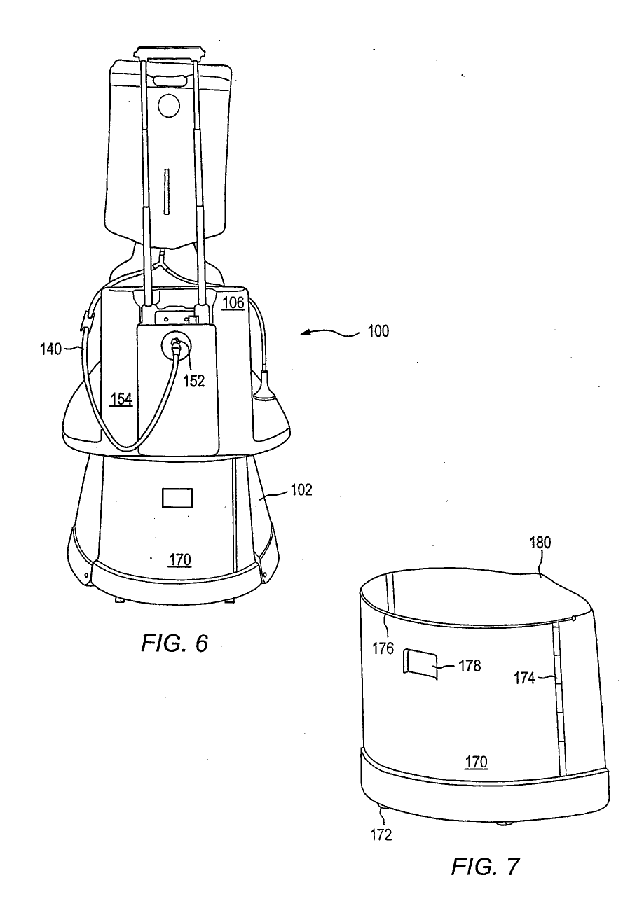

In some embodiments, a conduit is used to couple drain 166 of lavage chair 100 to a toilet or other waste acceptor. In certain embodiments, a waste container may be removably coupled to (e.g., positioned proximate to) a lavage chair such that effluent flowing through the drain of the lavage chair flows directly into the waste container. Removably coupling a waste container to a lavage chair allows use of the lavage chair in, for example, a hospital room or a bedroom. This may be advantageous for bedridden or mobility impaired users. FIG. 6 depicts an embodiment of lavage chair 100 removably coupled to waste container 170. In some embodiments, waste container 170 may be securely coupled to lavage chair 100 to inhibit loss of effluent from the waste container during use.

FIG. 7 depicts a perspective view of an embodiment of waste container 170. As shown in FIG. 7, waste container 170 may include wheels 172. Wheels 172 may be, for example, multi-directional caster wheels. Wheels 172 may allow an operator to roll waste container 170 from one location to another to dispose of effluent in the waste container. In some embodiments, waste container 170 includes window 174. Window 174 may be graduated to allow visual assessment of a volume of effluent in the waste container without uncoupling the waste container from lavage chair 100. In certain embodiments, waste container 170 may include handle 176 and/or grip 178 to facilitate rolling, carrying, and/or lifting the waste container. Spout 180 may facilitate pouring of effluent from waste container 170 into, for example, a toilet. In some embodiments, a removable cover (e.g., a snap-fit plastic lid) may be secured on waste container 170 to inhibit sloshing of effluent from the waste container while the waste container is being moved (e.g., rolled, carried, or lifted).

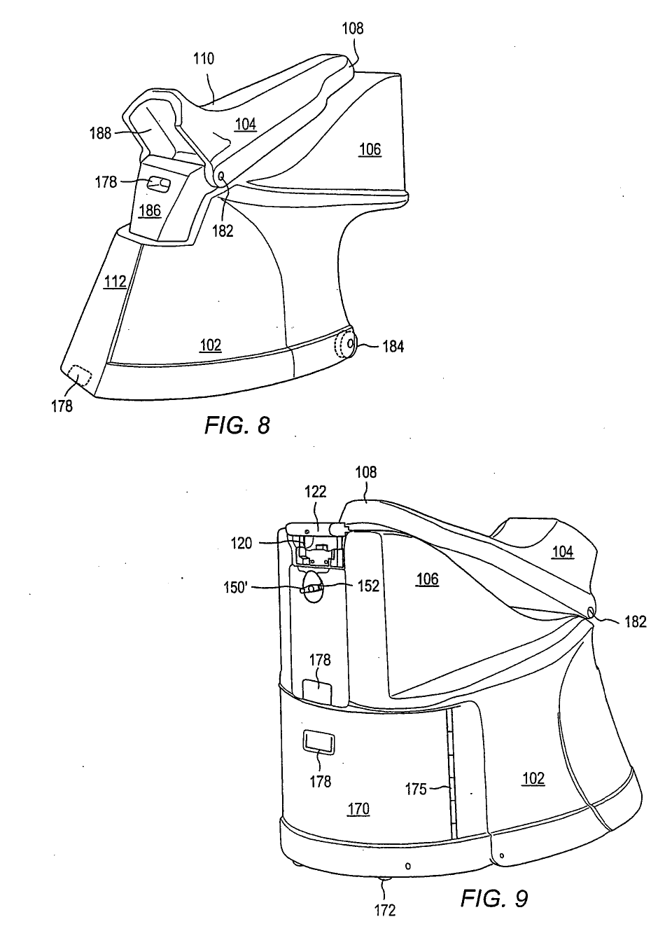

In some embodiments, a lavage chair may be reduced to achieve a more compact configuration of the lavage chair when not in use. For example, the lavage chair may be folded or disassembled to enhance portability and/or to reduce an amount of space required for storage. FIG. 8 depicts reduced lavage chair 100 with back 104 folded at rotatable connection 182 to facilitate storage and/or transportation of the lavage chair. As shown in FIG. 8, complementary regions of back 104 and base 102 proximate reinforced portions 110, 112 are designed to promote secure mating of the back and the base, thereby forming a stable support for the user. For example, portion 186 of base 102 may fit (e.g., snugly) in recess 188 of back 104 (as depicted in FIG. 1) to provide structural support to lavage chair 100 during use.

Lavage chair 100 may include wheels 184 to allow the lavage chair to be transported (e.g., pushed or pulled) easily. In some embodiments, lavage chair 100 includes one or more grips 178 sized and/or positioned to facilitate maneuvering or transporting (e.g., lifting, carrying) of the lavage chair. Grip 178 on portion 186 of base 102 may be exposed when back 104 is folded toward the front portion of the base. Thus, grip 178 on portion 186 is hidden during use of lavage chair 100, and is visible when needed to facilitate moving, lifting, and/or carrying the lavage chair. FIG. 9 depicts reduced lavage chair 100 coupled to waste container 170.

During transportation of lavage chair 100, extendable member 120 may be extended to allow an operator to roll the lavage chair as desired. For example, an operator may use handle 122 of extendable member 120 to tilt lavage chair 100, thereby decreasing an angle between front side 154 of the lavage chair, and roll (e.g., push or pull) the lavage chair to a desired location. Lavage chair 100 may be rolled with or without waste container 170 coupled to the lavage chair. After lavage chair 100 is positioned as desired, extendable member 120 may be retracted and locked in position for storage or extended and locked into position for use.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A lavage chair for a human, comprising:
a base;
a back connected to the base;
wherein the base and the back are configured to support a human in an at least partially supine position;
wherein the lavage chair is configured to be removably coupled to a toilet; and
wherein the lavage chair is configured to allow effluent to move from the colon of the at least partially supine human to the toilet during use.

2. (canceled)

3. The lavage chair of claim 1, wherein a portion of the back is configured to substantially mate with a portion of the base to provide support to the at least partially supine human.

4. The lavage chair of claim 1, wherein the lavage chair is configured to support the head of the at least partially supine human.

5. (canceled)

6. The lavage chair of claim 1, further comprising one or more footrests to support one or more legs and/or feet of the at least partially supine human.

7. The lavage chair of claim 1, further comprising a drain to allow the effluent to move from the colon of the at least partially supine human to the toilet.

8. The lavage chair of claim 1, further comprising an opening to allow the fluid to flow from a fluid source through a portion of the lavage chair to the colon of the at least partially supine human.

9-12. (canceled)

13. The lavage chair of claim 1, wherein the lavage chair is portable.

14. The lavage chair of claim 13, wherein the lavage chair comprises one or more grips configured to allow the lavage chair to be lifted.

15. The lavage chair of claim 13, wherein the lavage chair comprises one or more wheels configured to allow the lavage chair to be rolled along a surface.

16-24. (canceled)

25. The lavage chair of claim 1, wherein the
base is configured to rest on a surface; and
wherein the back is connected to the base at an angle of at least 10° between the back and the surface.

26. (canceled)

27. The lavage chair of claim 25, wherein the back is connected to the base at an angle between about 10° and about 80°.

28-30. (canceled)

31. The lavage chair of claim 25, wherein the back is rotatably connected to the base.

32. The lavage chair of claim 25, wherein the back is foldable from a first position which is at an angle of at least 10° to the surface to a second position wherein a portion of the back is closer to the toilet or to the waste container during use.

33. The lavage chair of claim 25, further comprising a seat portion.

34. The lavage chair of claim 25, further comprising one or more footrests for supporting one or more legs and/or feet of the human.

35. (canceled)

36. (canceled)

37. The lavage chair of claim 1, further comprising
an extendable member configured to support a fluid source at a height sufficient to allow fluid from the fluid source to enter the colon of the at least partially supine human during use.

38. (canceled)

39. The lavage chair of claim 37, wherein the extendable member is further configured to be used as a handle.

40. The lavage chair of claim 37, further comprising wheels, wherein the extendable member is further configured to be used as a handle, and wherein the lavage chair is rollable by an operator pushing or pulling on the handle.

41-43. (canceled)

44. The lavage chair of claim 37, wherein the fluid source is coupled to one or more conduits, and wherein at least one of the conduits is configured to be removably coupled to a portion of the lavage chair to allow fluid to flow from the fluid source, through a portion of the lavage chair, and into the colon of the at least partially supine human, and further comprising a valve and/or a regulator configured such that the rate of flow of the fluid from the fluid source is controllable by the at least partially supine human.

45. The lavage chair of claim 37, wherein the fluid source is coupled to one or more conduits, and wherein at least one of the conduits is coupled to a member configured to allow the at least partially supine human to rinse a portion of the user's body with fluid during use, and further comprising a valve and/or a regulator such that the rate of flow of the fluid used to rinse the portion of the user's body is controllable by the user.

46. (canceled)

47. (canceled)

48. (canceled)

49. The lavage chair of claim 1, further comprising
a front portion coupled to the base, wherein the front portion is located in front of an at least partially supine human during use, and the front portion comprises an opening configured to allow fluid from a fluid source to flow to the colon of the at least partially supine human during use.

50-55. (canceled)

56. The lavage chair of claim 49, wherein the lavage chair is configured such that fluid from the fluid source flows from a front side to the back side of the front portion during use.

57-60. (canceled)

61. A method of cleansing the colon or the vagina of a human, comprising:
positioning the human in an at least partially supine position on a stand-alone support;
coupling the support to a toilet;
allowing fluid to flow from a fluid source coupled to the support into the colon or the vagina through an orifice of the colon or the vagina;
allowing effluent to flow from the colon or the vagina through the orifice of the colon or the vagina into a portion of the support; and
allowing effluent to drain from the portion of the support into the toilet.

62. A method of cleansing the colon or the vagina of a human, comprising:
positioning the human in an at least partially supine position on a stand-alone support;
allowing fluid to flow through a portion of the support into the colon or the vagina through an orifice of the colon or the vagina;
allowing effluent to flow from the colon or the vagina through the orifice of the colon or the vagina into a portion of the support; and
allowing effluent to drain from the portion of the support into a waste container coupled to the support.
